(19) United States
(12) Patent Application Publication
(10) Pub. No.: US 2012/0258439 A1
(43) Pub. Date: Oct. 11, 2012

(54) Protein-Free Culture Media Products

(76) Inventor: Jaffar Ali bin M. Abdullah, Kuala Lumpur (MY)

(21) Appl. No.: 13/443,469

(22) Filed: Apr. 10, 2012

Related U.S. Application Data
(60) Provisional application No. 61/474,128, filed on Apr. 11, 2011.

(51) Int. Cl. A01N 1/02; C12N 5/071

(52) U.S. Cl. 435/1.1; 435/374

(57) ABSTRACT

Disclosed herein are compositions and methods for a substantially protein-free media (PFM) optimized for stem cell research.

### BACKGROUND OF THE INVENTION

1. Field of the Invention

The invention relates to a substantially protein-free media (PFM) optimized for stem cell research.

2. Description of Related Art

Previous claims of a chemically defined protein-free medium (PFM) for human applications, in fact, were not truly protein-free, because certain of such media contained serum proteins (Mohr & Trounson, 1986; Serta and Kiessling 1997 (Abstract); Parinaud et al. 1999). A totally protein-free media for stem cell research thus has not been previously described.

For example, a conventional commercially available culture media contain human serum albumin (HSA) obtained from human blood and tissue sources. In some laboratories bovine serum albumin (BSA) is also used as a source of protein (Loutradis et al., 1992; Quinn, 1994), similarly obtained from blood and tissue sources from cows. The efficiency of media containing HSA and BSA was reported to be similar (Staessen et al., 1998). The use in culture medium of protein obtained from donors (human or bovine) has the potential to transmit pathogenic diseases, in particular viral diseases, such as human acquired immunodeficiency disease syndrome (AIDS) and hepatitis, or Creutzfeldt-Jakob disease (CJD) transmitted by prions or others in blood-derived products has led a number of providers of healthcare services in the area of ART worldwide to seek alternative(s) to donor protein for their embryo culture and handling procedures. Therefore, there is a need in the art for a chemically defined medium for stem cell research.

The transmission of a deadly viral disease (AIDS) to hemophiliacs through blood-derived products is well documented (See, for example, Craven et al., 1997, Med. Sci. Law 37: 215-227; Keshavjee et al., 2001, Soc. Sci. Med. 53: 1081-1094; Weinberg et al., 2002, Ann. Intern. Med. 136: 312-319; Evatt, 2006, Semin. Hematol. 43: S4-9). Human growth hormone extracted from the pituitary was found to be capable of transmitting CJD to humans (Esmonde et al., 1994) and human gonadotropin injections could also transmit CJD from person to person (CDC, 1985). CJD can be transmitted through blood (although the titer of CJD prions is low in blood; Heye et al., 1994). In the past an epidemic of hepatitis B occurred in about 200 IVF patients that received embryos cultured in medium containing pooled sera contaminated with hepatitis B virus (van Os et al., 1991). Recently the scientific community was confronted with the dilemma of having to inform their patients that a commercial preparation of a culture medium used for embryo culture and handling may be contaminated with albumin donated by a person who later died of CJD (Kemmann, 1998).

In addition to its many biological roles, serum proteins confer useful physical attributes such as lubrication and viscosity in the culture medium. Increased viscosity and lubrication in the culture medium may be required for ease of handling and manipulation of, for example, the stem cell used in research.

The incorporation of PVP and PVA merely serve to duplicate the physical attributes of serum proteins. However, PVP and PVA are not sources of fixed nitrogen and they do not perform the various biological roles of proteins. In addition, the teratological properties of PVP and PVA have not been fully examined, which make their use for stem cell research.

The physiological functions of albumin and plasma proteins in general are well documented. The role of albumin in preventing membrane peroxidation indicates a direct role in membrane stability. It is involved in capillary membrane permeability and in osmoregulation. Albumin provides 80% of the total colloid osmotic pressure in plasma. Albumin is involved in the transport of carbon dioxide and acts as a pH buffer; albumin accounts for the greatest (95%) portion of the non-bicarbonate buffer value of plasma. Proteins also serve as a source of energy. Deaminated alanine is pyruvate, which can be either converted to acetyl-CoA or glucose and glycogen. Albumin may help solubilize lipids and transports hormones, vitamins and metals. It serves as reservoirs for the release and use of these components.

Any attempt at substituting serum albumin in culture medium should therefore take into consideration these in vivo roles and physical attributes. A single component may not fulfill all the functions of serum protein.

Although protein-free media that supports development of a number of animal species has been described previously, no such protein-free media has been successfully used in humans, nor could such media be presumed to support or be optimal for stem cell research. Therefore, there is a distinct need for a defined, protein-free growth medium especially adapted for stem cell research.

There are previously known putatively “protein-free” media for treating and cultivating mammalian cells, particularly cells from rodents (mice, rats, guinea pigs, etc.). Caro et al. “Protein-free” media for growth of mammalian or particularly human cells has been disclosed, inter alia, in Kovár et al., 1987, Biotechnology Letters, vol. 9 no. 4, p. 259-264 “Iron Compounds at high Concentrations Enable Hybridoma Growth in a Protein-free Medium”; Keen, 1995, Cytotechnology, vol. 17: 193-202 “The culture of rat myeloma and rat hybridoma cells in a protein-free medium”; Stoll et al., 1996, J. Biotechnology, vol. 45, p. 111-123 “Systematic improvement of a chemically defined protein-free medium for hybridoma growth and monoclonal antibody production.” Other publications disclosing protein-free growth media are: Zang et al., 1995, Biotechnology, vol. 13, p. 389-392, “Production of Recombinant Proteins in Chinese Hamster Ovary Cells Using A Protein-Free Cell Culture Medium”; and International patent application Publication No.: WO 2005/120576. Furthermore, the use of protein-free media (PFM) specialized and optimized for human reproduction and fertility programs has been previously disclosed in US Application Publication No.: 20090226879 and International patent application Publication No.: WO 2009086191.

### SUMMARY OF THE INVENTION

It is against the above background that the present invention provides certain advantages and advancements over the prior art.

Although the invention as set forth herein is not limited to specific advantages or functionalities, it is noted that in several embodiments the invention provide a substantially protein-free media (PFM) optimized for stem cell research. The present invention includes the formulation of a single medium solution that can replace and/or be used as a replacement/medium for all of the above media solutions. In exemplary embodiments of this invention, a series of substantially protein-free media are specifically disclosed. These media have the specific advantage of being of uniform composition devoid of potentially hazardous non-uniform biological components that may be harmful for stem cell research.

The completely defined nature of the substantially protein-free media formulations according to the present invention also is useful in facilitating stem cell research.

The substantially protein-free media of the invention may be stored frozen at −20° C. for up to 2 years without loss of efficacy.

The present invention may successfully overcome the need for added donor proteins in the culture system and may provide a substantially protein-free media system for stem cell research. The compositions, ranges, preferred ranges and particular specifications of the various components of the present invention are set forth herein. The present invention may be a product of studies on the effect, tolerance and determination of optimal levels of individual components such as amino acids, antioxidants and chelators, osmolytes, vitamins, nutrients and alternate energy sources that could substitute in part the various roles of protein in vivo and in vitro and which exemplifies the functions of proteins in various protein-free handling media (e.g., for use in stem cell research and/or for use as a stasis medium for stem cells, cell sheets on matrices, biopsies or short term storage of organs prior to transport). The optimal concentrations of the mentioned components may be utilized for stem cell research and/or for use as a stasis medium for stem cells, cell sheets on matrices, biopsies or short term storage of organs prior to transport. The present invention may thus successfully provide media useful for stem cell research and/or for use as a stasis medium for stem cells, cell sheets on matrices, biopsies or short term storage of organs prior to transport.

Disclosed herein is an optimized substantially protein-free cell culture medium for use, inter alia, as a stasis medium for stem cells, cell sheets on matrices, and biopsies or short term storage of organs prior to transport, the medium comprising mineral salts, amino acids, antioxidants, vitamins, nutrients, antibiotics, D-mannitol, and methylcellulose that has a molecular weight of 14,000 Daltons.

In further embodiments disclosed herein is a method comprising (i) providing a substantially protein-free cell culture medium comprising mineral salts, amino acids, antioxidants, vitamins, nutrients, antibiotics, D-mannitol, methylcellulose that has a molecular weight of 14,000 Daltons, and/or modifications; and (ii) using the substantially protein-free cell culture medium in stem cells, cell sheets on matrices, biopsies or short term storage of organs prior to transport.

Yet further, disclosed herein is a method of manufacturing a substantially protein-free cell culture medium for stem cells, cell sheets on matrices, biopsies or short term storage of organs prior to transport, comprising (i) identifying molecules required for maintaining stem cells in a quiescent, or resting, or for supporting stem cells in stasis, (ii)

identifying synthetic non-protein compounds that influence stasis while maintaining cells in culture, and (iii) confirming the effectiveness of the substantially protein-free cell culture medium for use as a stasis medium, as well as its ability to maintain the cells during long-distance transport.

In other embodiments, disclosed herein is a substantially protein-free cell culture medium for stem cells, cell sheets on matrices, biopsies or short-term storage of organs prior to transport manufactured as described herein.

It should be understood that in the methods and compositions described herein, the stem cells may comprise mammalian, such as human or animal, and/or avian stem cells. Furthermore, it should be understood that modified substantially protein free cell medium and substantially protein free cell medium with additional components may be used for stem cell research.

### DETAILED DESCRIPTION OF THE INVENTION

All publications, patents and patent applications cited herein are hereby expressly incorporated by reference for all purposes.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Before describing the present invention in detail, a number of terms will be defined. As used herein, the singular forms “a”, “an”, and “the” include plural referents unless the context clearly dictates otherwise.

The present invention provides a series of nutrient solutions that are devoid of any protein or protein-like components. These media are useful for, but not limited to, stem cell research, for example, for holding stem cells, cell sheets on matrices, biopsies and storage of organs prior to transport. In addition to the stem cell research, other methodologies and techniques where it would be advantageous to use substantially protein-free nutrient growth media include stem cell technology and therapy, cell/tissue regeneration and transplantation treatment procedures. The skilled worker will appreciate how to adapt the media set forth herein for such uses.

As used herein, the term “protein-free,” “essentially protein-free” and “substantially protein-free” is intended to mean that the media was prepared using non-protein containing components (as described in further detail herein) and that no protein or protein-containing components were added to the media.

Exemplary embodiments of the present invention include a series of substantially protein-free media for stem cell research.

These media are used according to the methods set forth below, which are intended to illustrate the use of these media but are not limiting to any additional uses of the media, inter alia, in stem cell research known to those with skill in the art. In developing the media of the invention in experiments as set forth herein, each procedure was performed in parallel using conventional protein-containing media and exemplary PFM of the invention.

PFM as a Stasis Medium

In certain aspects, the invention provides a substantially protein-free cell culture medium for use as a stasis medium for stem cells, cell sheets on matrices, biopsy tissue samples, or short-term storage of organs prior to transport.

In other aspects, the invention provides methods comprising methods to determine specific molecules required for maintaining stem cells in a quiescent, or resting, state by:

i. analyzing the applicability of the existing PF media formulations for supporting stem cells in statis,

ii. identifying specific, synthetic non-protein compounds that influence statis while maintaining cells in culture, and

iii. confirming the effectiveness of the PF media formulation for use as a stasis medium, as a well as its ability to maintain the cells during long-distance transport.

As used herein, the term “quiescent state,” “stasis,” or “resting state” refer to a state in which cells, such as stem cells, are not, at that time, undergoing repeated cell cycles, and therefore are not dividing. The ability to maintain their non-dividing condition (and in other terms, stay in the G-0 phase of cell cycle) is called quiescence. The G-0 phase is viewed as either an extended G-1 phase, where the cell is neither dividing nor preparing to divide, or a distinct quiescent stage that occurs outside of the cell cycle. Quiescent stem cells might be stimulated to undergo repeated cell cycles at a later time. Quiescence of stem cells is critical to ensure lifelong tissue maintenance and to protect the stem cell pool from premature exhaustion under conditions of various stresses. See, for example, Phinney (2010), Adult Stem Cells: Biology and Methods of Analysis, Springer, 279 pages; and Bosch (2008), Stem Cells: from hydra to man, Springer, 188 pages.

As used herein, a “stasis medium” is a cell culture medium that maintains cells, such as stem cells, in a static, quiescent, or resting state.

In some embodiments, the disclosed stasis media can be advantageously be used to maintain stem cells in a static or quiescent state in order to prevent their differentiation into unwanted cell types. Stem cells that remain in an undifferentiated state retain the ability to differentiate into a variety of cell types at a later time.

In other embodiments, the disclosed stasis media can be used to maintain cell sheets on matrices in a non-dividing condition. Such cell sheets may be used for tissue grafts or other tissue engineering purposes, and it may be desirable to prevent proliferation or differentiation of cells prior to the time at which the cell sheets are used for their intended purposes.

In yet other embodiments, the disclosed stasis media may be used to maintain biopsy tissue samples in a static or quiescent state, such that the samples do not change with regard to cellular composition or other properties from the time at which the samples are taken to the time at which the samples are analyzed. In this case, a stasis medium would help ensure that the biopsy tissue samples were more representative of a patient's condition than if the cells within the samples were to undergo cell division or differentiation after being removed from the patient, but before analysis.

In yet other embodiments, the disclosed stasis media may be used for the short-term storage of organs prior to and during transport and prior to transplantation or other intended uses. In this manner, the cells within the organs may be preserved in a static, yet viable state, without undergoing unwanted cell differentiation or division.

Media Formulation

The formulation of an exemplary substantially protein-free media of the invention described herein was as follows. Albumin is known to be a source of fixed nitrogen and nutrients, an antioxidant, and to also have a number of other roles including membrane stabilization. Thus, the substantially protein-free media of the invention substituted albumin with other media components that perform, individually or collectively, the functions of albumin in culture. Individual PFM culture medium components used for these purposes include amino acids (including but not limited to alanine, asparagine, aspartate, cystine, glutamate, glutamine, glycine, histidine, isoluecine, leucine, lysine, methionine, phenylalanine, taurine, thereonine, tryptophan, tyrosine, valine, serine), antioxidants and chelators (for example, EDTA, reduced glutathione, tocopherol), alternate energy sources (such as fructose, glutamine, sodium pyruvate), osmolytes (including mannitol and myoinositol), vitamins (ascorbic acid, cyanocobalamin, folic acid, tocopherol, etc) and elemental iron.

The final formulations for the various media products of the invention are given in tables as set forth below. The concentrations of HEPES and bicarbonate in these media preparations vary, as set forth herein.

The substantially protein-free media of the invention comprise mineral salts, amino acids, antioxidants, antibiotics, energy components and buffer components (HEPES and bicarbonate for incubation under CO₂-supplemented conditions) that are similar to but distinct in their particulars from commercially-available, protein-containing media. The substantially protein-free media of the invention uniquely comprise a macromolecular species (methylcellulose and related polymers) and an optionally an un-metabolized sugar alcohol (D-mannitol).

The macromolecule comprising the substantially protein-free media of the invention is methyl cellulose of Formula I:

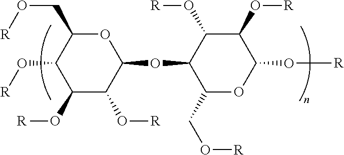

wherein each R is independently CH₃ or H and n is between about 34 and about 43.

In the formula of methylcellulose above, R can be either H or CH₃. The extent of methoxy substitution ranges between 27.5-31.5% by weight. Degree of substitution (D.S., average number of substituent groups attached to the ring hydroxyls) is 1.5-1.9.

This range gives maximum water solubility. Lower methoxy substitution results in higher solubility in water and is preferred. The code “n” designates the level of polymerization, and optimally corresponds to a molecular weight of 14,000 Daltons relating to an approximate viscosity index of 15 cPS for a 2% solution in water at 20° C. (methylcellulose having these specifications is commercially available from Sigma Chemical Co., St. Louis, Mo. USA; www.sigmaaldrich.com); this corresponds to a value of “n” that is between about 34 and about 43, preferably 36 to 39. The range of this component in the PFM is 0.01 to 0.15 g/L and the most preferred range is 0.09 to 0.1 g/L. The optimal concentration is 0.1 g/L.

Methylcellulose can act as an antioxidant and osmolyte, it is non-toxic, enzyme resistant and not cell permeable (Stewart et al., 1995). It may help to protect stem cell used in research against environmental insults, in particular attack by free radicals and osmotic pressure changes. It may protect the cellular membrane from damage and helps maintain homeostasis. It is also a surfactant and lubricant. It contributes to increased viscosity of the medium, so that the stem cell used in research do not stick to sides of dishes and inside pipettes and catheters. These physical attributes are supplied in the absence of serum proteins that normally perform these functions. This substance is inert and safe for human application.

Methylcellulose has been used elsewhere in human pharmaceutical and food industry for well over 25 years without any side effects in human or animal studies. FAO/WHO and EU directives allow consumption of methylcellulose, and it has been used as a negative control in cancer research as it is known to be non-carcinogenic.

Methylcellulose is also used as a thickener and emulsifier in various food and cosmetic products, and has medicinal uses, such as for treatment of constipation. It is not digestible, non-toxic, and not allergenic. Its pharmacological/clinical uses are as excipients and a carrier material. It is used in eye drops, as a bulking agent and laxative, used for diarrhea in functional bowel disease, to control ileostomy output and as absorbent of toxic substances that causes infective diarrhea. It is also an antioxidant.

The substantially protein-free media of the invention also comprises D-mannitol. This compound is poorly absorbed and is excreted almost unchanged in urine. As an antioxidant and an osmolyte this substance may protect stem cell used in research from harmful environmental effects. It thus can further substitute for serum proteins, in part, to protect stem cell used in research against adverse conditions. It is of interest to note that D-mannitol exerts a positive effect on mouse blastocyst development in vitro even in the presence of protein in the media.

In the exemplary substantially protein-free media of this invention, D-mannitol is present at a preferred concentration of 2.8 micromolar (the most preferred concentration) within the range of 0.056 to 6.9 micromolar. The more preferred range is 1.4 to 5.5 micromolar; even more preferred within the range of 2.5 to 3.0 micromolar. The most preferred concentration is about 2.7 micromolar. The empirical formula of D-mannitol is C₆H₁₄O₆ and has the structural formula:

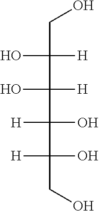

D-mannitol is a food additive, used in cakes, confectioneries and sweets; being sweeter than sucrose, it is considered an alternative sweetener for diabetics. It is an osmolyte and antioxidant. It has been used in high concentrations to treat acute stroke for well over 30 years. Hypermolar concentrations of this compound are used to treat severe brain damage and elevated intracranial pressure. It is a diuretic and is used for dieresis in instances of poisoning, or to measure extracellular fluid compartment. It also has laxative effects in mammals including man. No adverse effects in man have been reported as a result of clinical application of D-mannitol as a therapeutic agent. It is also non-cytotoxic and non-mutagenic in several species. D-mannitol is poorly absorbed and is excreted (Milde, 1965; Widdowson and Dickerson, 1965). Its use is permitted by the FDA and the EU, and FAO/WHO has concluded it to be safe for human consumption.

In the solutions and formulations of the invention, D-mannitol of Formula II is present at a concentration of from about 0.05 micromolar to about 6.9 micromolar. The more preferred range is 1.4 to 5.5 micromolar. The most preferred concentration is about 2.8 micromolar.

The skilled worker will appreciate that the role of serum proteins are numerous, and in its absence, one or two components by themselves may not provide a complete substitute for proteins in the medium. Thus, the skilled worker will recognize that the invention does not merely provide media supplemented with methylcellulose and D-mannitol. Rather, the invention provides media comprising in addition a complex mixture of additional components, preferably in optimal concentrations, and the selective exclusion of commonly used media components that have proven detrimental to stem cells. Some of these include:

(i) Including unique concentrations for two amino acids (L-taurine and glutamine) that are the principal providers of nutrition and osmotic balance. These amino acids are provided at concentrations within the range of 1 mM to 30 mM L-taurine and 1 mM to 50 mM L-glutamine, more preferably 10 mM to 30 mM L-taurine and 10 mM to 30 mM L-glutamine and most preferably 20 mM each of L-taurine and L-glutamine.

(ii) Including as energy sources any one or plurality of compounds (including D-glucose, fructose, or pyruvate). Optimal concentrations of fructose are from about 0.5 mM to 6.0 mM fructose, with a more preferred concentration being from about 1 mM to 5.6 mM, and a most preferred concentration of about 5.1 mM. The optimal concentrations of the remaining two energy sources are given elsewhere in this document.

(iii) Including the following concentrations of certain amino acids.

| | | Preferred | Most |
|---|---|---|---|
| | Optimum range | range | preferred |
| Name | Conc. in PFM (mM) | (mM) | Conc. (mM) |
| L-alanine | 0.1-10 | 0.45-0.55 | 0.5 |
| L-arginine | 0.018-0.18 | 0.072-0.125 | 0.072 |
| L-cystine•2HCl | 0.0025-0.025 | 0.01-0.02 | 0.01 |
| L-glutamate | 0.01-1.0 | 0.45-0.55 | 0.5 |
| L-glycine | 0.1-1.0 | 0.2-0.3 | 0.25 |
| L-histidineHCl•H₂O | 0.005-0.05 | 0.02-0.04 | 0.02 |
| L-isoleucine | 0.01-0.1 | 0.04-0.08 | 0.04 |
| L-leucine | 0.01-0.1 | 0.04-0.08 | 0.04 |
| L-lysine HCl | 0.0125-0.125 | 0.05-0.1 | 0.05 |
| L-methionine | 0.0025-0.025 | 0.01-0.02 | 0.01 |
| L-phenylalanine | 0.005-0.05 | 0.02-0.04 | 0.02 |
| L-threonine | 0.01-0.1 | 0.04-0.08 | 0.04 |
| L-tryptophan | 0.00125-0.0125 | 0.005-0.01 | 0.005 |
| L-tyrosine•2Na2H2O | 0.005-0.05 | 0.02-0.04 | 0.02 |
| L-valine | 0.01-0.1 | 0.04-0.08 | 0.04 |

(iv) Including the following concentrations of water-soluble vitamins.

| | | Preferred range | Most |
|---|---|---|---|
| | Optimum range | Conc. | preferred |
| Name | (mM) | (mM) | Conc. (mM) |
| Choline chloride | 0.004-0.007 | 0.004-0.005 | 0.004 |
| D-Biotin | 0.0024-0.004 | 0.0024-0.003 | 0.0024 |
| Folic acid | 0.0014-0.0023 | 0.0014-0.0016 | 0.0014 |
| Myoinositol | 0.0067-0.011 | 0.0067-0.0078 | 0.0067 |
| Niacinamide | 0.005-0.008 | 0.005-0.0057 | 0.005 |
| D-pantothenic Acid | 0.0025-0.004 | 0.0025-0.003 | 0.0025 |
| ½Ca | | | |
| Pyridoxine HCl | 0.003-0.005 | 0.003-0.0034 | 0.003 |
| Riboflavin | 0.00016-0.00027 | 0.00016-0.00019 | 0.00016 |
| Thiamine HCl | 0.0018-0.003 | 0.0018-0.002 | 0.0018 |

The range for vitamin B12 is as follows:

| | | Preferred range | Most preferred |
|---|---|---|---|
| | Optimum range | Conc. | Conc. in PFM |
| Name | (pM) | (pM) | (pM) |
| Vitamin B12 | 443-885 | 590-738 | 616 |

(v) Including vitamin E (Vitamin E Type 6, Sigma Chemical Co.) at a concentration of from 5 micromolar to 20 micromolar, more preferably 8 micromolar to 12 micromolar, even more preferably 10 micromolar.

(vi) In some embodiments, the PFM may exclude L-asparagine, L-aspartate and L-serine. In some embodiments, the PFM may contain L-asparagine, L-aspartate and L-serine.

(vii) In some embodiments, the PFM may exclude elemental iron. In some embodiments, the PFM may contain elemental iron.

(viii) Including reduced glutathione (GSH) at a concentration of 60 micromolar to 500 micromolar, and more preferably 250 micromolar to 350 micromolar, and even more preferably 300 micromolar.

Other Compounds

### Concentration in Final Solution

| | | | Most | |
|---|---|---|---|---|
| | | Preferred | Preferred | |
| Description | Range | Range | Conc. | |
| D-glucose | 0.75-1.0 | 0.75-0.90 | 0.78 | g/L |
| Sodium chloride | 6.12-6.95 | 6.12-6.19 | 6.171 | g/L |
| Potassium chloride | 0.35-0.4 | 0.35-0.36 | 0.355 | g/L |
| Calcium chloride- | 0.23-0.27 | 0.23-0.24 | 0.235 | g/L |
| Magnesium sulfate | 0.086-0.098 | 0.086-0.087 | 0.087 | g/L |
| Na-dihydrogen | 0.107-0.122 | 0.107-0.109 | 0.108 | g/L |
| phosphate | | | | |
| Na-EDTA | 0.0416-0.043 | 0.0416-0.042 | 0.0418 | g/L |
| Sodium bicarbonate* | 2.2 | 2.2 | 2.2 | g/L |
| Sodium lactate, | 1.9 | 1.9 | 1.9 | ml/L |
| 60% syrup | | | | |
| Phenol red | 0.011 | 0.011 | 0.011 | g/L |

*Sodium bicarbonate
2.2 g/L (26.2 mM) 2.2 g/L (26.2 mM) 2.2 g/L (26.2 mM)

HEPES

In some embodiments, the PFM may contain from about 0 mM to about 25 mM Hepes.

The PFM set forth herin differs from conventional culture media in at least the following aspects:
Absense of adding donor serum proteins to media.
The presence of methylcellulose and D-mannitol.
Alterations in the composition of the media regarding species and concentrations of amino acids, antioxidants, vitamins, energy sources and mineral salts, all optimized for the stem cell used in research.

Among the advantages of the media of the invention are reduced risks to persons handling the PFM. While certain risks remain these are minor in comparison with the infectious risks avoided using the substantially protein-free media of the invention. These risks include allergies to the components used. The chance of this occurring is very unlikely because the ingredients except for antibiotics are mostly inert and non-reactive. All ingredients are in minute concentrations not likely to elicit an allergic response.

Preparation of PFM of the Invention

Basal Salt Solution (BSS) Stock Solution 1:

Preparation of stock solution of basal salts (Basal Salt Solution—BSS) to be used is a final formulation of PFM Culture medium.

| | | Quantity | |
|---|---|---|---|
| | Raw material component | (g/L) | |
| 1. | Calcium chloride•2H₂O | 2.65 | (23.8 mM) |
| 2. | Magnesium sulphate (anhyd.) | 0.9767 | (8.1 mM) |
| 3. | Potassium chloride | 4.0 | (53.7 mM) |
| 4. | Sodium chloride | 69.53 | (1.2M) |
| 5. | Sodium phosphate monobasic | 1.22 | (10 mM) |
| | (anhyd.) | | |
| 6. | D-glucose | 10.517 | (58.4 mM) |
| 7. | Phenol red | 0.11 | (0.31 mM) |

| | | |
|---|---|---|
| 8. | WFI (Water for injection) | 950 mL |

Preparation procedure: 1. Rinse mixing container with WFI (Water for injection, 18.2 MegaOhms resistivity) before preparation of stock solution. 2. Add component 1 to 1000 mL of WFI water as it is extremely hydroscopic. 3. Add components 2-7 in order, mixing continually and make up final volume to 1000 mL using WFI. 4. Sterile filtration to be carried out immediately after all solutes are fully dissolved. Do not filter if solutes remain. 0.1 micron filters can be used but avoid excessive pressure when filtering. The Stock 1 solution can be pre-filtered with 0.2 micron filter followed by 0.1 micron filter. 5. There should be no precipitate or cloudiness post-filtration. 6. Fill into containers of suitable volume, e.g. bottles. 7. Cap bottles (preferably tamper-evident seal bottles). 8. Store in the dark between 2 and 6 degrees Celsius.

Storage and Shelf Life.

The Basal Salt Solution (BSS) stock was stable for two months when stored between 2 and 6 degrees Celsius. Always check stored BSS stock carefully for precipitates or cloudiness before use. Discard if precipitates occur or the solution has turned cloudy during storage.

Inclusion Volumes of BSS Stock 1 in Final Product:

Where sodium bicarbonate is the predominantly active buffer component in the formulation (Culture media products), 70 ml** of BSS must be present in every 1000 ml of final formulation prepared.

Where HEPES is the predominantly active buffer component in the formulation (sperm/flushing media products), 50 mL of BSS is added for every 1000 mL of final formulation prepared.

Adjusting the osmolality of final medium with BSS or WFI water to increase or decrease (respectively) the osmolality of the medium:

Should the osmolality of the final medium be 285 mOsmols when final volume is less than 1000 ml (i.e. after adding BSS, BAAS and BVS); then separately dilute a small amount of BSS with WFI. Use 1 volume of BSS with 9 volumes of WFI water to give a working BSS (WBSS). Adjust osmolality of WBSS to 285 mOsmols. Make up final volume of final medium to 1000 ml with adjusted WBSS.

For composition of final formulation from all stock solutions—see Stock 4 instructions.

Basal Amino Acid Solution (BAAS) Stock Solution 2:

This protocol was used to prepare a 1 liter stock solution of basal amino acids in solution (Basal 5 Amino Acid Solution—BAAS) for inclusion in final formulations of PFM Culture medium.

Reagents

| | | Quantity |
|---|---|---|
| | Raw material component | (g/L) |
| 1. | L-arginine HCl | 1.26 (6.0 mM) |
| 2. | L-cystine (L cystine•2HCl)* | 0.313 (1.0 mM) |
| 3. | L-histidine HCl•H2O | 0.42 (2.0 mM) |
| 4. | L-isoleucine | 0.52 (4.0 mM) |
| 5. | L-leucine | 0.52 (4.0 mM) |
| 6. | L-lysine•HCl | 0.752 (4.1 mM) |
| 7. | L-methionine | 0.15 (1.0 mM) |
| 8. | L-phenylalanine | 0.32 (1.9 mM) |
| 9. | L-threonine | 0.48 (4.0 mM) |
| 10. | L-tryptophan | 0.1 (0.49 mM) |
| 11. | L-tyrosine (L-tyrosine•2Na 2H₂O)* | 0.519 (2.0 mM) |
| 12. | L-valine | 0.46 (3.9 mM) |
| 13. | WFI (Water for injection) | 1000 mL |

Instructions for Preparation of Stock 2 BAAS

1. Rinse mixing containers with WFI (Water for injection, 18.2 MegaOhms resistivity) 2. Dissolve components 1, 2 and 4-11 in 400 mLs of WFI.

Compliance with Supplier's storage recommendations is required as difficulties with solubility may be experienced with less than optimal conditions. If a precipitate occurs discard and start again using 25 mL of 0.11 N NaOH and 275 mL of WFI. Sodium hydroxide is only used as last option.

3. L-histidine HCl—H₂O and L-valine can be difficult to dissolve and in such circumstances solubility can be achieved by using 1N HCl. Components 3 and 12 should be dissolved in their own separate 300 mL volumes of WFI. Use only the smallest volume possible of 1N HCl to achieve solubilization. The maximum amount of 1N HCl that should be used is 25 mL in 300 mL of WFI. Avoid heating water, alkalis and acids above 37 degrees Celsius to dissolve all components.

4. Once components 3 and 12 are fully dissolved, make up final volume of 1000 mL by gradually adding in a stepwise manner, a 200 mL portion of the water soluble amino acids made in step 2 to: (a) 300 mL solution of L-histidine HCl.H₂O giving 500 mL in total volume. (b) 300 mL solution of L-valine giving 500 mL in total volume.

5. Combine both 500 mL volumes, gradually in a stepwise manner with constant mixing to avoid precipitation.

6. Sterile filter solution with 0.2 micron filter immediately

7. Fill into containers of suitable volume (e.g., bottles).

8. Cap bottles (preferably tamper-evident seal bottles).

9. Store in the dark between 2-6 degrees Celsius.

Storage and Shelf Life.

The Basal Amino Acid Solution (BAAS) stock will keep for two months when stored between 2 and 6 degrees Celsius. Always check stored BAAS stock carefully for precipitates or cloudiness before use. Discard if precipitates or cloudiness occurs during storage.

Inclusion Volumes of BAA Stock in Final Product:

10 ml of BAA stock solution should be used per 1000 ml in the preparation of all final formulations. L-Tyrosine.2Na 2H₂O and L-Cystine.2HCl were used because free form L-Tyrosine and L-Cystine were not commercially available.

Basal Vitamin Solution (BVS) Stock Solution 3.

The protocol according to this example prepares a 1 liter stock solution of basal vitamins in solution (Basal Vitamin Solution—BVS) for inclusion in final formulations of PFM Culture medium.

Reagents

| | Raw material component | Quantity (g/L) | |
|---|---|---|---|
| 1. | Choline chloride | 0.1 | (0.7 mM) |
| 2. | D-biotin | 0.1 | (0.4 mM) |
| 3. | Myoinositol | 0.2 | (1.1 mM) |
| 4. | Niacinamide | 0.1 | (0.82 mM) |
| 5. | D-pantothenic acid | 0.1 | (0.46 mM) |
| 6. | Pyridoxine HCl | 0.1 | (0.49 mM) |
| 7. | Riboflavin | 0.01 | (26.5 μM) |
| 8. | Thiamine HCl | 0.1 | (0.33 mM) |
| 9. | Folic acid | 0.1 | (0.23 mM) |

| | | |
|---|---|---|
| 10. | WFI (Water for injection) | 1000 mL |

Instructions for Preparation of Stock 3 (BVS)

1. Dissolve components 1-7 in 800 mL of WFI water (Water for injection, 18.2 MegaOhms resistivity). Should any component not dissolve, small amounts of 0.1 N or 1 N NaOH can be used to achieve solvation as a last option.

2. Dissolve component 8 in 2.5 mL of 0.1 N NaOH and add carefully in a stepwise manner, with constant mixing to 800 mL containing components 1-7.

3. Make up volume to 1000 mL using WFI water with constant mixing and sterile filter immediately with 0.2 micron filter into suitable containers (e.g., 60 mL Nalgene bottles, tamper-evident bottles).

4. Cap bottles.

5. Store in the dark at −20 degrees Celsius.

Storage and Shelf-Life.

The Basal Vitamin Solution (BVS) stock can be stored for two months when stored at −20 degrees Celsius in 60 mL aliquots. Always check thawed BVS stock 3 carefully for precipitates or cloudiness before use. Discard if precipitates appear or the thaw solution appears cloudy.

Inclusion Volumes of BVS Stock into Final Product:

6.0 mL of BVS stock solution should be used per 1000 mL in the preparation of all final formulations prepared—see Stock 4.

Stock 4 Solution and Final Formulation Combinations.

The preparation of Stock 4 permits the final addition of the unique chemicals in this Formulation and the addition of specific volumes taken from Stock 1 Basal Salts Solution (BSS), Stock 2 Basal Amino Acid Solution (BAAS) and Stock 3 Basal Vitamin Solution (BVS). The protocol describes the addition of buffers and antibiotics that are specific to the media formulation being prepared.

The following protocol prepares 1 liter of final formulation of Culture medium. Larger batches may be prepared by appropriate scaling of quantities.

Reagents

| | Raw material | | |
|---|---|---|---|
| | component | Quantity (g/l) | |
| 1. | Sodium pyruvate | 0.02975 | (0.27 mM) |
| 2. | Fructose | 0.92125 | (5.1 mM) |
| 3. | Glycine | 0.01875 | (0.25 mM) |
| 4. | Glutathione | 0.09225 | (0.30 mM) |
| | (Reduced) | | |
| 5. | D-mannitol | 0.0911 | (0.5 mM) |
| 6. | EDTA (sodium | 0.04175 | (0.10 mM) |
| | tetra) | | |
| 7. | L-alanine | 0.04455 | (0.5 mM) |
| 8. | L-taurine | 1.251 | (1.0 mM) 20 mM |
| 9. | L-glutamic acid | 0.0735 | (0.39 mM) |
| | mono sodium | | |
| 10. | Lactic acid | 1.9 | mL per liter |
| | Sodium salt | | |
| 11. | Vitamin B-12 | 100 | μL/L |
| 12. | Vitamin E Type 6 | 333 | μL/L |
| 13. | Stock 1 BSS | 70 | mL |
| 14. | Stock 2 BAAS | 10 | mL |
| 15. | Stock 3 BVS | 6 | mL |

| | | Quantity g/L | Quantity g/L | Quantity g/L |
|---|---|---|---|---|
| | | Culture medium | Culture medium | Culture medium |
| 16. | Gentamycin | 1.5-4 | | 1.5-4 |
| | sulphate | | | |
| 17. | Penicillin G | 75 | 75 | |
| 18. | L-glutamine | 1.461 (20 mM) | 1.461 (20 mM) | 1.461 (20 mM) |
| 19. | Sodium bicarbonate | 2.2 (26.2 mM) | 2.2 (26.2 mM) | 2.2 (26.2 mM) |
| 20. | HEPES | 0.0 | 0.0 | 0.0 |
| 21. | Methyl cellulose | 0.1 | 0.1 | 0.1 |

| | | | |
|---|---|---|---|
| 22. | WFI (Water for | 1000 | mL |
| | injection) | | |

In further embodiments, the substantially protein-free cell culture medium suitable for use as a stasis medium for stem cells, cell sheets on matrices, biopsies or short term storage of organs prior to transport may comprise 0.1 g/L (0.0071 mM) methylcellulose having a molecular weight of 14,000 Daltons; 0.5 mM L-arginine; 0.01 mM L-cystine.2HCl; 0.02 mM L-histidine, 0.04 mM L-isoleucine; 0.04 mM L-leucine; 0.05 mM L-lysine.HCl; 0.01 mM L-methionine; 0.02 mM L-phenylalanine; 0.04 mM L-threonine; 0.005 mM L-tryptophan; 0.02 mM L-tyrosine.2Na2H₂O; 0.04 mM L-valine; 0.5 mM L-alanine; 20 mM for L-taurine; 0.5 mM glutamic acid (0.39 mM); 20 mM L-glutamine or 0.25 mM L-Glycine; 3.1 mM (0.235 g/L) calcium chloride; 0.72 mM (0.087 g/L) magnesium sulfate; 4.8 mM (0.355 g/L) potassium chloride; 0.11 M (6.171 g/L) sodium chloride; 0.88 mM (0.108 g/L) for sodium dihydrogen phosphate; 0.004 mM (0.0005 g/L) choline chloride; 0.0024 mM (0.0006 g/L) D-biotin; 0.0067 mM (0.0012 g/L) myoinositol; 0.005 mM (0.0006 g/L) niacinamide; 0.0025 mM (0.0005 g/L) D-pantothenic acid; 0.003 (0.0006 g/L) pyridoxine HCl; 0.00016 mM (0.00006 g/L) riboflavin; 0.0018 mM (0.0005 g/L) thiamine HCl; 0.0014 mM (0.006 g/L) folic acid; 616 pM (800 ng/L) vitamin B12; 0.010 mM (0.004 g/L) vitamin E; between about 4.2 mM (0.75 g/L) to about 5.6 mM (1.0 g/L) D-glucose; 0.3 mM (0.02975 g/L) sodium pyruvate; 5.1 mM (0.92 g/L) fructose; 10 mM (1.13 g/L) sodium lactate; 0.3 mM (0.092 g/L) glutathione; 0.1 mM (0.0418 g/L) EDTA; 0.031 mM (0.011 g/L) phenol red; 2.8 micromolar D-mannitol; 26.2 mM (2.2 g/L) sodium bicarbonate; and 75 mg/L Penicillin G or from about 1.5 mg/L to about 4 mg/L gentamycin sulfate.

The substantially protein-free cell culture medium suitable for use as a stasis medium for stem cells, cell sheets on matrices, biopsies or short term storage of organs prior to transport may further comprise the concentration of gentamycin sulfate of 4 mg/L and the concentration of D-glucose is between about 4.2 mM (0.75 g/L) to about 5.6 mM (1.0 g/L) and further comprising 15 mM (3.5745 g/L) HEPES. The concentration of D-glucose may be between about 4.2 mM (0.75 g/L) to about 5.6 mM (1.0 g/L) and may further comprise 25 mM (5.9575 g/L) or 15 mM (3.5745 g/L) HEPES.

The substantially protein-free cell culture medium suitable for use as a stasis medium for stem cells, cell sheets on matrices, biopsies or short term storage of organs prior to transport may further comprise the concentration of gentamycin sulfate is 1.5 mg/L and the concentration of D-glucose is between about 4.2 mM (0.75 g/L) to about 5.6 mM (1.0 g/L) and further comprising 15 mM (3.5745 g/L) HEPES. The concentration of D-glucose may be between about 4.2 mM (0.75 g/L) to about 5.6 mM (1.0 g/L) and may further comprise 25 mM (5.9575 g/L) or 15 mM (3.5745 g/L) HEPES.

Instructions for Preparation of Stock 4 and Final Formulation Combination.

1. Rinse mixing vessel with WFI (Water for injection, 18.2 MegaOhms resistivity).

2. Dissolve components 1-11 in 700 mL of WFI mixing continuously whilst the additions are made.

3. Add components 12-15 and make up to 900 mL with WFI and continue mixing.

4. Add components 16-21 depending on preparation of the basic formulation type i.e., PFM Culture media.

5. Adjust osmolality of all three media and separately filter sterilize using 0.2 microns pore-size filters, 0.1 micron filter can also be used but avoid using pressure. Do not use 0.1 microns filter if high pressure required for filtering the solution.

6. Sterile filter immediately into final packaging (Nalgene bottles): PFM Culture media is filled into 60 mL bottles. (Bottles used are preferably tamper-evident bottles.)

7. Cap bottles.

8. Label bottles.

9. Store in the dark at between 2 and 6 degrees Celsius.

Footnote for Stock 4 and Final Formulation Combination.

Check osmolality. If the osmolality is high, then adjust to 285 mOsmols by adding WFI pure water. The amount to be added is calculated as follows:

[Osmolality of medium−Desired osmolality(i.e.,285)/Osmolality of medium]×volume of medium

### Example

If osmolality of medium is 300 and volume of medium is 900 ml as above, then calculate as shown below:

[300−285/300]×900=15/300×900=45

Therefore if you add 45 ml of water to the medium and then measure osmolality again, you should theoretically have an osmolality of about 285 plus/minus 2 or 3 units. Be very careful not to add too much water because the important ingredients in the medium will become diluted and will affect the efficacy of the medium, even if you bring back the osmolality of the medium by adding more Stock 1 BSS solution.

However, if osmolality is lower than 285 mOsmols, add Stock 1 BSS only to the medium, approximately 2 to 3 mOsmols increases per ml of Stock 1 BSS but this may not always be predictable. So be very careful not to add too much. Add a little at a time and measure osmolality.

It should be understood that, as compared to the PFM for human reproduction and fertility programs previously disclosed in US Application Publication No.: 20090226879 and International patent application Publication No.: WO 2009086191, the PFM medium of the invention may be optimized for stem cell research. It should be understood that the PFM medium of the invention may contain additional components, such as buffers, salts, amino acids, and other components. It should further be understood that the PFM medium of the invention may not contain all of the components described above. Furthermore, it should be understood that additional steps may be taken to prepare the PFM medium of the invention.

### REFERENCES

Ali J. (1997) Formulation of a protein-free culture system for the culture of human embryos: preliminary findings and pregnancies. In: Programme and abstract book of the 16th Annual Scientific meeting of the Fertility Society of Australia, 2-4 Dec. 1997, Adelaide, Australia, p34 (Abstract).
Ali J. (2000) Investigation into the nutrient requirement of the human embryos: Successful formulation and clinical trial of a novel protein-free embryo culture medium. Emirates Med J. 18:195-202.
Ali, J (2004) Generation of viable human embryos in a protein-free embryo culture (ART-7b) medium enhances clinical pregnancy rate and prevents disease transmission in assisted reproduction. Middle East Fertil Soc. J. 9:118-127.
Ali, J., Abdulkader, A., Shahata, M. A. M. et al. (1998) Term deliveries from human embryos conceived in a novel culture medium devoid of added protein. Middle East Fertil. Soc. J. 3, 40 (Abstract).
Ali, J., Whitten, W. K. and Shelton, J. N. (1993). Effect of culture systems on mouse early embryo development. Hum. Reprod. 8, 1110-1114.
Almagor, M., Bejar, C., Kafka, I. et al. (1996) Pregnancy rates after communal growth of pre-implantation human embryos in vitro. Fertil. Steril. 66, 394-397.
Balakier, H. (1993) Tripronuclear human zygotes: the first cell cycle and subsequent development. Hum. Reprod. 8, 1892-1897.
Barber, A. A. (1961) Inhibition of lipid peroxide formation by vertebrate blood serum. Arch. Biochem. Biophys. 92, 38-43.
Barnes, R I, Crombie, A., Gardner, D. K. et al. (1995) Blastocyst development and pregnancy after in vitro maturation of human primary oocytes, intracytoplasmic sperm injection and assisted hatching. Hum. Reprod. 10, 3243-3247.
Bavister, B. D. (1995) Culture of pre-implantation embryos: facts and artifacts. Hum. Reprod. Update 1, 91-148.
Biggers, J. D., Summers, M. C. and McGinnis, K. L. (1997) Polyvinyl alcohol and amino acids as substitutes for bovine serum albumin in culture media for mouse pre-implantation embryos. Hum. Reprod. Update 3, 125-135.
Boyers, S. P., Diamond, M. P., Lavy, G. et al. (1987) The effect of polyploidy on embryo cleavage after in vitro fertilization in humans. Fertil. Steril. 48, 624-627.
Canesco, R. S., Sparks, A., Pearson, R. E. et al. (1992) Embryo density and medium volume effects on early marine embryo development. J. Assist. Reprod. Genet. 9, 454-457.
Caro, C. M. and Trounson, A. (1986) Successful Fertilization, Embryo Development, and Pregnancy in Human in Vitro Fertilization (IVF) Using a Chemically Defined Culture Medium Containing No Protein. Journal of in Vitro Fertilization and Embryo Transfer, vol. 3, no. 4. p. 215-217).
Carrillo, A. J., Lane, 13., Pridham, D. D. et al. (1998) Improved clinical outcomes for in vitro fertilization with delay of embryo transfer from. 48 to 72 hours after oocyte retrieval: use of glucose- and phosphate-free media. Fertil. Steril. 69, 329-334 Centers for Disease Control. (1985) Fatal degenerative neurologic disease in patients who received pituitary derived human growth hormone. MMWR Morb Moral Wkly Rep 34:359-60, 365-6.
Cholewa, J. A. and Whitten, W. K. (1965) Development of 2-cell mouse embryos in the absence of a fixed nitrogen source. J. Reprod. Fertil. 22, 553-555.
Cockle S M, Aitken A, Beg F, Morrell J M, Smyth D G. (1989) The TRH-related peptide by pyroglutamylglutamylprolinamide is present in human semen. FEBS Lett 1989; 252:13-7
Dandekar, P. V. and Glass, R. H. (1990) Development of two-cell mouse embryos in protein-free and protein-supplemented media. J. In Vitro Fertil. Embryo Transfer 7, 107113.
De Mouzon, J. and Lancaster, P. (1997). World collaborative report on in vitro fertilization. Preliminary data for 1995. J. Assist. Reprod. Genet. (suppl.) 14, 250S-265S.
Drakakis, P., Loutradis, D., Milingos, S. et al. (1996) The in vitro development of mouse embryos beyond the blastocyst stage into the hatching and outgrowth stage using different energy sources. J. Assist. Reprod. Genet. 13, 786-792.
Du, Z. F. and Wales, R. G. (1993) Effect of culture from the zygote stage on the metabolism of glucose and glutamine by 2-cell embryos and blastocysts recovered from. outbred or H female mice. Reprod. Fertil. Dev. 5, 555-565.
Esmonde T, Lueck C J D, Symon L, Duchen L W, Will R G. (1994) Creutzfeldt-Jakob disease and lyophilised dura mater grafts: report of two cases. J. Neurol Neurosurg Psychiat. 56:999-1000.
Gardner, D. K. (1994) Mammalian embryo culture in the absence of serum or somatic cell support. Cell Biol. Int. 18, 1163-1179.
Gardner, D. K. and Lane, M. (1998a) Culture of viable human blastocysts in defined sequential serum-free media. Hum. Reprod. (suppl.) 13, 148-160.
Gardner, D. K., Schooleraft, W. B., Wagley, L. et al. (1998b) A prospective randomized trial of blastocyst culture and transfer in in-vitro fertilization. Hum. Reprod. 13, 3434-3440.
Green C M, Cockle S M, Watson P P et al. (1996) A possible mechanism of action for fertilization promoting peptide, a TRH-related tripeptide that promotes capacitation and fertilizing ability in mammalian spermatozoa. Mol. Reprod. Dev, 45:244-52.
Heyc, N., Henson, S., and Muller, N. (1.994) Creutzfeldt-Jakob disease and blood transfusion. Lancet 343:298299.
Kane, K T., Morgan, P. M. and Coonan, C. (1.997) Peptide growth factors and preimplantation development. Hum. Reprod. Update 3:137-157.
Kemmann, E. (1998) Creutzfeldt-Jakob disease (CJD) and assisted reproductive technology (ART). Hum. Reprod. 13, 1777.
Lane, M. and Gardner, D. K. (1997) Non-essential amino acids and glutamine decrease the time of the first three cleavage divisions and increase compaction of mouse zygotes in vitro. J. Assist. Reprod. Genet. 14, 398-403.
Li, J., Foote, R. H., Liu, Z. et al. (1996) Development of rabbit zygotes into blastocysts in defined protein-free medium and offspring born following culture and embryo transfer. Theriogenology 47, 1103-1113.
Lighten, A. D., Moore, G I., Winston, et al. (1998). Routine addition of human insulinlike growth factor-1 ligand could benefit clinical in-vitro fertilization culture. Hum. Reprod. 13, 3144-3150.
Loutradis, D., Kallianidis, K., Drakkis, P. et al. (1992) Successful pregnancy in human IVF using BSA as a protein source in the transfer medium. Assist. Reprod. Technol. Androl. 3, 233-238.
Mehta, T. S. and Kicssling, A. A. (1990) Development potential of mouse embryos conceived in vitro and cultured in ethylenediaminetetraacetic acid with and without amino acids or serum. Biol. Reprod. 43, 600-606.
Milde, M. D. (1965). Ann. Rev. Pharmac. 5: 125.
Moessner, J. and Dodson, W. C. (1993) The role of growth factors and proteins in media for in vitro fertilization. Assist. Reprod. Rev. 3, 63-67.
Palermo, G., Joris, H., Devroey, P. and Van Steirteghem, A (1992) Pregnancies after intracytoplasmic injection of a single spermatozoon into oocyte. Lancet. 340:17-18.
Paria, B. C. and Dey, S. K. (1990) Pre-implantation embryo development in vitro: cooperative interactions among embryos and the role of growth factors. Proc. Natl. Acad. Sci. USA, 87, 4756-4760.
Paria, B. C, Tsukamura, H. and Iley, S. K. (1991) Epidermal growth factor-specific protein tyrosine phosphorylation in pre-implantation embryo development. Biol. Reprod. 45, 711-718.
Parinaud, J., Milhet, P., Vieitiez, G. et al. (1998a) Human sperm capacitation and in vitro fertilization in a chemically defined and protein-free medium (SMART1). Proceedings of the 16th World Congress on Fertility and Sterility and the 54th Annual Meeting of the American Society for Reproductive Medicine. Oct. 4-9, 1998, San Francisco, Calif., United States of America, Fertil. Stern. (suppl.) p.S195-S196 (Abstract).
Parinaud, J., Vieitiez, Milhet, P., et al. (1998b) Use of plant enzyme preparation (coronase) instead of hyaluronidase for cumulus cell removal before intracytoplasmic sperm injection. Hum. Reprod. 13, 1933-1935.
Poiley, S. M. (1960) A systematic method of breeder rotation for non-inbred laboratory animal colonies. Proc. Anim. Care Panel, 10, 159-166.
Quinn, P. (1994) Use of co-culture with cumulus cells in insemination medium in human in vitro fertilization (IVF). J. Assist. Reprod. Genet. 11, 270-277.
Saito, H., Hirayama, T., Koike, K. et al. (1994) Cumulus mass maintains embryo quality. Steril. 62, 555-558.
Schramm, R. D. and Bavister, B. D. (1996) Development of in-vitro-fertilized primate embryos into blastocysts in a chemically defined, protein-free culture medium. Hum. Reprod. 11, 1690-1697.
Serta, R. S., Sakellariou, M., Kiessling, A. A. et al. (1997) Outcome of human embryos conceived and cleaved in protein-free culture conditions. Proceedings of the 53^{rd} Annual Meeting of the American Society for Reproductive Medicine. October 1.
Stewart G J, Wang Y, Niewiarowski S. (1995) Methylcellulose protects the ability of anchorage-dependant cells to adhere following isolation and holding in suspension. Biotechniques. 19(4):598-604.
Meeting of the American Society for Reproductive Medicine. October 188-22, 1997, Cincinnati, Ohio, United States of America, (suppl.) 5214-5215 (Abstract).
Spindle, A. (1995) Beneficial effect of taurine on mouse zygotes developing in protein-free culture medium. Theriogenology 44, 761-772.
Staessen, C., Janssenswillen, C., De Clerck, E. et al. (1998) Controlled comparison of commercial media for human in-vitro fertilization: Menezo B2 medium versus Medi-Cult universal and BM1 medium. Hum. Reprod. 13, 2548-2554.
Tesarik, J., Pilka, L., Drahorad, J. et al. (1988) The role of cumulus cell-secreted proteins in the development of human sperm fertilizing ability: implications in 1VF. Hum. Reprod. 3, 129-132.
Parainaud, J., Milhet, P., Vieitez, G. and Richoilley, G. (1999). Use of a medium devoid of 5 any human or animal compound (SMART2I) for embryo culture in intracytoplasmic sperm injection. J. Assist. Reprod. Genet. 16: 13-16.
Truyen, U., Parrish, C. R., Harder, T. C. et al. (1995) There is nothing permanent except change. The emergence of new viral diseases. Vet. Microbiol. 43, 103-122. Cited in: Parinaud et al., 1998b.
Van Os, H. C., Drogendijk, Aat, C. Fetter, W. P. F. et al. (1991) The influence of contamination of culture medium with hepatitis B virus on the outcome of in vitro fertilization pregnancies. Am. J. Obstet. Gynecol. 165, 152-159.
Vidlakova, Erazimova, J., Horky, J. et al. (1972) Relationship of serum antioxidative activity to tocopherol and serum inhibitor of lipid peroxidation, Chim. Acta 36, 61-66.
Wayner, Dam, Burton, G. W., Ingold, K. U. et al. (1987) The relative contribution of vitamin F, mate, ascorbate and proteins to total peroxyl radical trapping antioxidant activity of human blood plasma. Biochim. Biophys. Acta 924, 408-419.
Widdowson, E. M. and Dickerson, J. W. T. (1964). In: Comar, L. C. and Brommer, F. (eds.), Mineral Metabolism, New York—London, Vol. IIA, p. 13.

We claim:

1. An optimized substantially protein-free cell culture medium for use as a stasis medium for stem cells, cell sheets on matrices, biopsies or short term storage of organs prior to transport, the medium comprising mineral salts, amino acids, antioxidants, vitamins, nutrients, antibiotics, D-mannitol, and methylcellulose that has a molecular weight of 14,000 Daltons.

2. The substantially protein-free medium according to claim 1, wherein said methylcellulose is characterized so that a 2% solution has a viscosity of 15 centipoise at 25° C.

3. The substantially protein-free medium according to claim 1, wherein said methylcellulose is of formula I: 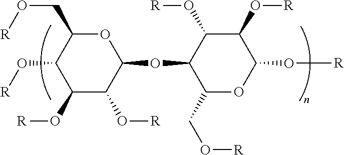
wherein each R is independently H or CH₃ and n is an integer having a value from about 34 to about 43 and wherein methoxy substitution is from 27.5% to 31.5% by weight.

4. The substantially protein-free medium according to claim 3, wherein the average number of CH₃ substituents attached to each sugar moiety of the compound of formula I is 1.5 to 1.9.

5. The substantially protein-free medium according to claim 1, 2, 3, or 4, wherein said methylcellulose is present in the solution at a concentration from 0.01 g/L (0.71 micromolar) to 0.5 g/L (0.036 mM).

6. The substantially protein-free medium according to claim 1, 2, 3, or 4, wherein said methylcellulose is present in the solution at a concentration from 0.01 g/L (0.71 micromolar) to 0.15 g/L (0.00011 mM).

7. The substantially protein-free medium according to claim 1, 2, 3, or 4, wherein said methylcellulose is present in the solution at a concentration of about 0.1 g/L (0.0071 mM).

8. The substantially protein-free medium according to claim 1, 2, 3, or 4, wherein the amino acids are L-arginine, L-cystine, L-histidine, L-isoleucine, L-leucine, L-lysine, L-methionine, L-phenylalanine, L-threonine, L-tryptophan, L-tyrosine, L-valine, L-alanine, L-taurine, L-glutamic acid, L-glutamine or glycine, or any combination thereof.

9. The substantially protein-free medium according to claim 8, wherein the amino acids are present at concentrations between: about 0.018 mM and about 0.18 mM for L-arginine HCl; about 0.0025 mM and about 0.025 mM for L-cystine.2HCl; about 0.005 mM and about 0.05 mM for L-histidine HCl.H₂O; about 0.01 mM and about 0.1 mM for L-isoleucine; about 0.01 mM and about 0.1 mM for L-leucine; about 0.0125 mM and about 0.125 mM for L-lysine.HCl; about 0.0025 mM and about 0.025 mM for L-methionine; about 0.005 mM and about 0.05 mM for L-phenylalanine; about 0.01 mM and about 0.1 mM for L-threonine; about 0.00125 and about 0.0125 mM L-tryptophan; about 0.005 and about 0.05 mM L-tyrosine.2Na2H₂O; about 0.01 mM and about 0.1 mM for L-valine; about 1.0 mM and about 10 mM for L-alanine; about 1.0 mM and about 30 mM for L-taurine; about 0.01 mM and about 1.0 mM for glutamic acid; about 1.0 mM and about 50 mM for L-glutamine or about 0.1 mM and about 1.0 mM for L-glycine.

10. The substantially protein-free medium according to claim 9, wherein the amino acids are present at a concentration of: 0.5 mM L-arginine; 0.01 mM L-cystine.2HCl; 0.02 mM L-histidine, 0.04 mM L-isoleucine; 0.04 mM L-leucine; 0.05 mM L-lysine.HCl; 0.01 mM L-methionine; 0.02 mM L-phenylalanine; 0.04 mM L-threonine; 0.005 mM L-tryptophan; 0.02 mM L-tyrosine.2Na2H₂O; 0.04 mM L-valine; 0.5 mM L-alanine; 20 mM for L-taurine; 0.5 mM glutamic acid; and 20 mM L-glutamine or 0.25 mM L-Glycine.

11. The substantially protein-free medium according to claim 1, 2, 3, or 4, wherein the mineral salts comprising the medium are calcium chloride, magnesium sulfate, potassium chloride, sodium chloride and sodium phosphate.

12. The substantially protein-free medium according to claim 11, wherein the mineral salts comprising the medium are present at concentrations between: about 3.0 mM (0.23 g/L) and 3.6 mM (0.27 g/L) for calcium chloride; about 0.7 mM (0.086 g/L) and about 0.81 mM (0.098 g/L) for magnesium sulfate; about 4.7 mM (0.35 g/L) and about 5.4 mM (0.4 g/L) for potassium chloride; about 0.1 M (6.12 g/L) and about 0.12 M (6.95 g/L) for sodium chloride; and about 0.89 mM (0.107 g/L) to about 1.0 mM (0.122 g/L) for sodium dihydrogen phosphate.

13. The substantially protein-free medium according to claim 12, wherein the mineral salts comprising the medium are present at a concentration of: 3.1 mM (0.235 g/L) for calcium chloride; 0.72 mM (0.087 g/L) for magnesium sulfate; 4.8 mM (0.355 g/L) for potassium chloride; 0.11 M (6.171 g/L) for sodium chloride; and 0.88 mM (0.108 g/L) for sodium dihydrogen phosphate.

14. The substantially protein-free medium according to claim 1, 2, 3, or 4, wherein the vitamins comprising the medium are choline chloride, myoinositol, niacinamide, D-pantothenic acid, pyridoxine HCl, riboflavin, thiamine HCl, folic acid, vitamin B12, vitamin E, or any combination thereof.

15. The substantially protein-free medium according to claim 14, wherein the vitamins comprising the medium are present at concentrations between: about 0.004 mM (0.0005 g/L) and 0.005 mM (0.0007 g/L) for choline chloride; about 0.0024 mM (0.0006 g/L) and 0.0028 (0.0007 g/L) D-biotin, about 0.0067 mM (0.0012 g/L) and 0.0078 mM (0.0014 g/L) for myoinositol; about 0.005 mM (0.0006 g/L) and 0.0057 mM for niacinamide (0.0007 g/L); about 0.0025 mM (0.0005 g/L) and 0.003 mM (0.0007 g/L) for D-pantothenic acid; about 0.003 (0.0006 g/L) mM and 0.0034 mM (0.0007 g/L) for pyridoxine HCl; about 0.00016 mM (0.00006 g/L) and 0.00019 mM (0.00007 g/L) for riboflavin; about 0.0018 mM (0.0005 g/L) and 0.0021 mM (0.0006 g/L) for thiamine HCl; about 0.0014 mM (0.006 g/L) and 0.0016 mM (0.0007 g/L) for folic acid; about 443 pM (600 ng/L) and 885 pM (1.2 mg/L) for vitamin B12; and 0.008 mM (0.003 g/L) and 0.012 mM (0.005 g/L) for vitamin E.

16. The substantially protein-free medium according to claim 15, wherein the vitamins comprising the medium are present at a concentration of 0.004 mM (0.0005 g/L) for choline chloride; 0.0024 mM (0.0006 g/L) D-biotin; 0.0067 mM (0.0012 g/L) for myoinositol; 0.005 mM (0.0006 g/L) for niacinamide; 0.0025 mM (0.0005 g/L) for D-pantothenic acid; 0.003 (0.0006 g/L) for pyridoxine HCl; 0.00016 mM (0.00006 g/L) for riboflavin; 0.0018 mM (0.0005 g/L) for thiamine HCl; 0.0014 mM (0.006 g/L) for folic acid; 616 pM (800 ng/L) for vitamin B12; and 0.010 mM (0.004 g/L) vitamin E.

17. The substantially protein-free medium according to claim 1, 2, 3, or 4, wherein the nutrients comprising the medium are D-glucose, pyruvate, fructose, lactic acid, or any combination thereof.

18. The substantially protein-free medium according to claim 17, wherein the nutrients comprising the solution are present at a concentration of 4.3 mM (0.78 g/L) for D-glucose; 0.3 mM (0.02975 g/L) for sodium pyruvate; 5.1 mM (0.92 g/L) for fructose; and 10 mM (1.13 g/L) for sodium lactate.

19. The substantially protein-free medium according to claim 18, wherein D-glucose is present at a concentration between 4.2 mM (0.75 g/L) and 5.6 mM (1.0 g/L).

20. The substantially protein-free medium according to claim 19, wherein fructose is present at a concentration between 1 mM (0.18 g/L) and 5.6 mM (1.01 g/L).

21. The substantially protein-free medium according to claim 1, 2, 3, or 4, wherein the antioxidant in the solution is glutathione.

22. The substantially protein-free medium according to claim 21, wherein the concentration of glutathione is between about 0.25 mM (0.077 g/L) and 0.35 mM (0.11 g/L).

23. The substantially protein-free medium according to claim 22, wherein the concentration of glutathione is 0.3 mM (0.092 g/L).

24. The substantially protein-free medium according to claim 1, 2, 3, or 4, further comprising EDTA.

25. The substantially protein-free medium according to claim 24, wherein the concentration of EDTA is between about 0.1 mM (0.0416 g/L) and 0.103 mM (0.043 g/L).

26. The substantially protein-free medium according to claim 25, wherein the concentration of EDTA is 0.1 mM (0.0418 g/L).

27. The substantially protein-free medium according to claim 1, 2, 3, or 4, further comprising HEPES.

28. The substantially protein-free medium according to claim 27, wherein the concentration of HEPES is 15 mM (3.5745 g/L) or 25 mM (5.9575 g/L), and the concentration of D-Glucose is between about 4.2 mM (0.75 g/L) to about 5.6 mM (1.0 g/L).

29. The substantially protein-free medium according to claim 1, 2, 3, or 4, further comprising phenol red or other pH indicator.

30. The substantially protein-free medium according to claim 29, wherein the concentration of phenol red is 0.031 mM (0.011 g/L).

31. The substantially protein-free medium according to claim 1, 2, 3, or 4, wherein gentamycin sulfate is present in the solution at a concentration from about 1.5 mg/L to about 4 mg/L.

32. The substantially protein-free medium according to claim 1, 2, 3, or 4, wherein Penicillin G is present at a concentration of 75 mg/L.

33. The substantially protein-free medium according to claim 1, 2, 3, or 4, wherein D-mannitol is present at a concentration from about 0.056 micromolar to about 6.9 micromolar.

34. The substantially protein-free medium of claim 33, wherein D-mannitol is present at a concentration of 2.8 micromolar.

35. The substantially protein-free medium of claim 1, 2, 3, or 4, further comprising sodium bicarbonate.

36. The substantially protein-free medium of claim 35, wherein sodium bicarbonate is present at a concentration of 26.2 mM (2.2 g/L).

37. The substantially protein-free medium of claim 36, wherein sodium bicarbonate is present at a concentration of between 4.0 mM (0.336 g/L) and 26.2 mM (2.2 g/L).

38. The substantially protein-free cell culture medium for stem cell research according to claim 1, prepared from at least one or a plurality of stock solutions of mineral salts, amino acids, antioxidants, vitamins, nutrients, antibiotics, D-mannitol, and methylcellulose having a molecular weight of 14,000 Daltons, wherein said stock solutions are diluted with water to form the substantially protein-free cell culture medium.

39. A substantially protein-free cell culture medium suitable for use as a stasis medium for stem cells, cell sheets on matrices, biopsies or short term storage of organs prior to transport, comprising 0.1 g/L (0.0071 mM) methylcellulose having a molecular weight of 14,000 Daltons; 0.5 mM L-arginine; 0.01 mM L-cystine.2HCl; 0.02 mM L-histidine, 0.04 mM L-isoleucine; 0.04 mM L-leucine; 0.05 mM L-lysine.HCl; 0.01 mM L-methionine; 0.02 mM L-phenylalanine; 0.04 mM L-threonine; 0.005 mM L-tryptophan; 0.02 mM L-tyrosine.2Na2H₂O; 0.04 mM L-valine; 0.5 mM L-alanine; 20 mM for L-taurine; 0.5 mM glutamic acid (0.39 mM); 20 mM L-glutamine or 0.25 mM L-Glycine; 3.1 mM (0.235 g/L) calcium chloride; 0.72 mM (0.087 g/L) magnesium sulfate; 4.8 mM (0.355 g/L) potassium chloride; 0.11 M (6.171 g/L) sodium chloride; 0.88 mM (0.108 g/L) for sodium dihydrogen phosphate; 0.004 mM (0.0005 g/L) choline chloride; 0.0024 mM (0.0006 g/L) D-biotin; 0.0067 mM (0.0012 g/L) myoinositol; 0.005 mM (0.0006 g/L) niacinamide; 0.0025 mM (0.0005 g/L) D-pantothenic acid; 0.003 (0.0006 g/L) pyridoxine HCl; 0.00016 mM (0.00006 g/L) riboflavin; 0.0018 mM (0.0005 g/L) thiamine HCl; 0.0014 mM (0.006 g/L) folic acid; 616 pM (800 ng/L) vitamin B12; 0.010 mM (0.004 g/L) vitamin E; between about 4.2 mM (0.75 g/L) to about 5.6 mM (1.0 g/L) D-glucose; 0.3 mM (0.02975 g/L) sodium pyruvate; 5.1 mM (0.92 g/L) fructose; 10 mM (1.13 g/L) sodium lactate; 0.3 mM (0.092 g/L) glutathione; 0.1 mM (0.0418 g/L) EDTA; 0.031 mM (0.011 g/L) phenol red; 2.8 micromolar D-mannitol; 26.2 mM (2.2 g/L) sodium bicarbonate; and 75 mg/L Penicillin G or from about 1.5 mg/L to about 4 mg/L gentamycin sulfate.

40. The substantially protein-free cell culture medium of claim 39, wherein the concentration of gentamycin sulfate is 4 mg/L.

41. The substantially protein-free cell culture medium of claim 39, wherein the concentration of D-glucose is between about 4.2 mM (0.75 g/L) to about 5.6 mM (1.0 g/L) and further comprising 15 mM (3.5745 g/L) HEPES.

42. The substantially protein-free cell culture medium of claim 39, wherein the concentration of D-glucose is between about 4.2 mM (0.75 g/L) to about 5.6 mM (1.0 g/L) and further comprising 25 mM (5.9575 g/L) HEPES.

43. The substantially protein-free cell culture medium of claim 40, wherein the concentration of D-glucose is between about 4.2 mM (0.75 g/L) to about 5.6 mM (1.0 g/L) and further comprising 15 mM (3.5745 g/L) HEPES.

44. The substantially protein-free cell culture medium of claim 40, wherein the concentration of D-glucose is between about 4.2 mM (0.75 g/L) to about 5.6 mM (1.0 g/L) and further comprising 25 mM (5.9575 g/L) HEPES.

45. The substantially protein-free cell culture medium of claim 39, wherein the concentration of gentamycin sulfate is 1.5 mg/L.

46. The substantially protein-free cell culture medium of claim 45, wherein the concentration of D-glucose is between about 4.2 mM (0.75 g/L) to about 5.6 mM (1.0 g/L) and further comprising 15 mM (3.5745 g/L) HEPES.

47. The substantially protein-free cell culture medium of claim 46, wherein the concentration of D-glucose is between about 4.2 mM (0.75 g/L) to about 5.6 mM (1.0 g/L) and further comprising 25 mM (5.9575 g/L) HEPES.

48. A method comprising,
(i) providing the substantially protein-free cell culture medium of claim 1 or 39, comprising mineral salts, amino acids, antioxidants, vitamins, nutrients, antibiotics, D-mannitol, methylcellulose that has a molecular weight of 14,000 Daltons, and/or modifications; and
(ii) using the substantially protein-free cell culture medium in stem cells, cell sheets on matrices, biopsies or short term storage of organs prior to transport.

49. A method of manufacturing a substantially protein-free cell culture medium of claim 1 or 39, for stem cells, cell sheets on matrices, biopsies or short term storage of organs prior to transport, comprising,
(i) identifying molecules required for maintaining stem cells in a quiescent, or resting, or for supporting stem cells in stasis,
(ii) identifying synthetic non-protein compounds that influence stasis while maintaining cells in culture, and
(iii) confirming the effectiveness of the substantially protein-free cell culture medium for use as a stasis medium, as well as its ability to maintain the cells during long-distance transport.

50. The substantially protein-free cell culture medium for stem cells, cell sheets on matrices, biopsies or short term storage of organs prior to transport manufactured by the method of claim 49.
